(19) United States
(12) Patent Application Publication
(10) Pub. No.: US 2012/0232480 A1
(43) Pub. Date: Sep. 13, 2012

(54) Retractable Syringe with Conical Frustum Tip

(76) Inventor: Richard Caizza, Vernon, NJ (US)

(21) Appl. No.: 13/413,559

(22) Filed: Mar. 6, 2012

Related U.S. Application Data
(60) Provisional application No. 61/449,966, filed on Mar. 7, 2011.

(51) Int. Cl. A61M 5/50; A61M 5/34

(52) U.S. Cl. 604/110; 604/240; 604/241

(57) ABSTRACT

A retractable needle syringe having a conical frustum tip configured to selectively attach to a Luer device including an inexpensive commonly found Luer filling needle, and also including an interchangeable needle assembly configured to secure to a needle stem positioned within the conical frustum tip. The conical frustum tip advantageously facilitates aspirating a medicant, using a common Luer needle for filling and also prevents the possible unintentional retraction of the needle assembly due to hydraulic pressures encountered when drawing the medicant from a vial and during the handling of the syringe. Moreover, the conical frustum tip advantageously allows the syringe to be conveniently prefilled with a medicant at a pharmacy and capped, then transported to a patient with or without needle assembly as desired. This design is a significant advantage for many healthcare workers involved in the processing and handling of syringes until ultimate delivery of the medicant to a patient.

### CROSS REFERENCE TO RELATED APPLICATIONS

This application claims priority of U.S. Provisional Patent Application Ser. No. 61/449,966 entitled Retractable Syringe with Luer Tip, filed Mar. 7, 2011, the teachings of which are incorporated herein.

### BACKGROUND OF THE INVENTION

The present invention is directed to safety syringes, and particularly syringes with retractable needles configured to retract after use and prevent re-use and unintended sticking. Retractable syringes are known in the art, which typically include a needle configured to automatically retract into a syringe barrel after delivery of a medicant into a patient. The syringes typically have a fixed needle, meaning different sized needles cannot be selectively interchanged with a syringe barrel. These standard fixed needle arrangements are configured to draw the medicant from a vial, which process can inadvertently damage the needle point while attempting to insert the needle into the vial, or during the handling process. Damaged needle points cause pain when inserted and withdrawn from patients and are undesirable.

There is desired a retractable syringe configured to draw a medicant with one large bore needle and inject the patient with another smaller bore needle, and preferably an interchangeable needle assembly.

### BRIEF DESCRIPTION OF THE FIGURES

FIG. 1 shows an exploded view of a retractable syringe having a conical frustum tip, as well as an interchangeable needle and hub forming a needle assembly configured to extend through the conical frustum tip;

FIG. 2 shows a longitudinal cross sectional view of the syringe of FIG. 1 depicting a needle stem positioned in the syringe distal end and configured to receive the interchangeable needle assembly disposed through the conical frustum tip;

FIG. 3 shows an enlarged sectional view of the syringe distal end illustrating the needle stem having a threaded distal end configured to receive needles of various sizes through the conical frustum tip;

FIG. 4 shows a longitudinal cross sectional view of the syringe of FIG. 1 depicting the needle assembly after retraction into the syringe barrel; and

FIG. 5 shows a cross section of the syringe barrel just distal of the segmented retaining ledge shown in FIG. 3.

### SUMMARY OF THE INVENTION

The present invention achieves technical advantages as a retractable needle syringe having a conical frustum tip configured to selectively attach to female Luer devices such as valves and fittings, a Luer filling needle, and also including an interchangeable needle assembly configured to secure to a needle stem to prevent liquid flow between surfaces positioned within the conical frustum tip. The Luer filling needle is advantageous as a readily available inexpensive needle type found in medical facilities. Large bore needles speed up drawing a medicant, and also prevents the possible unintentional retraction due to hydraulic pressures encountered within the syringe body when drawing a particularly highly viscous medicant and during the handling of the syringe. Moreover, the conical frustum tip advantageously allows the syringe to be conveniently prefilled with a medicant using a fill needle at a pharmacy or filling station and the needle removed and the syringe safely capped, then transported to a patient with or without needle assembly as desired. This design is a significant advantage for many healthcare personnel involved in the processing and handling of syringes until ultimate delivery of the medicant to a patient.

### DETAILED DESCRIPTION OF A PREFERRED EMBODIMENT

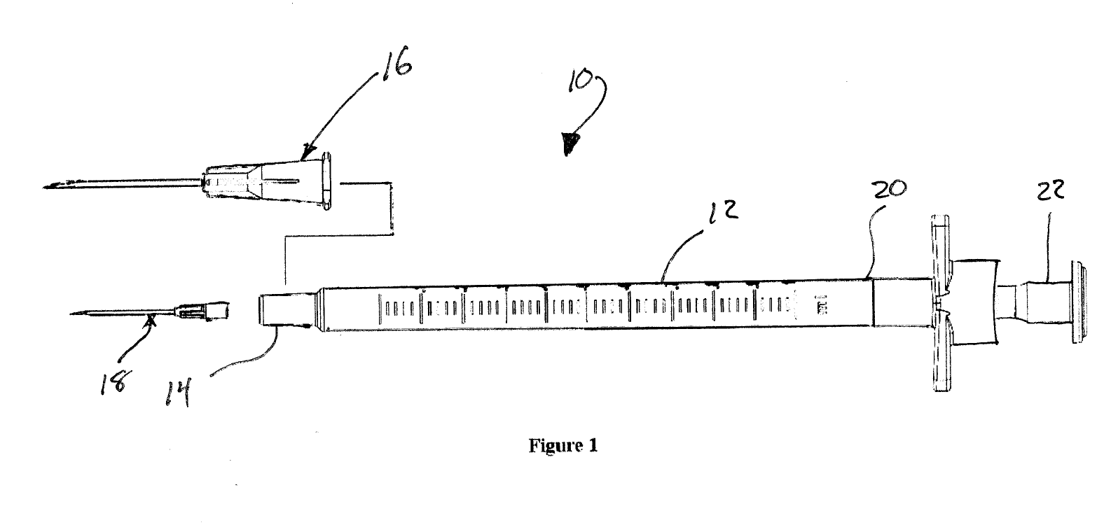

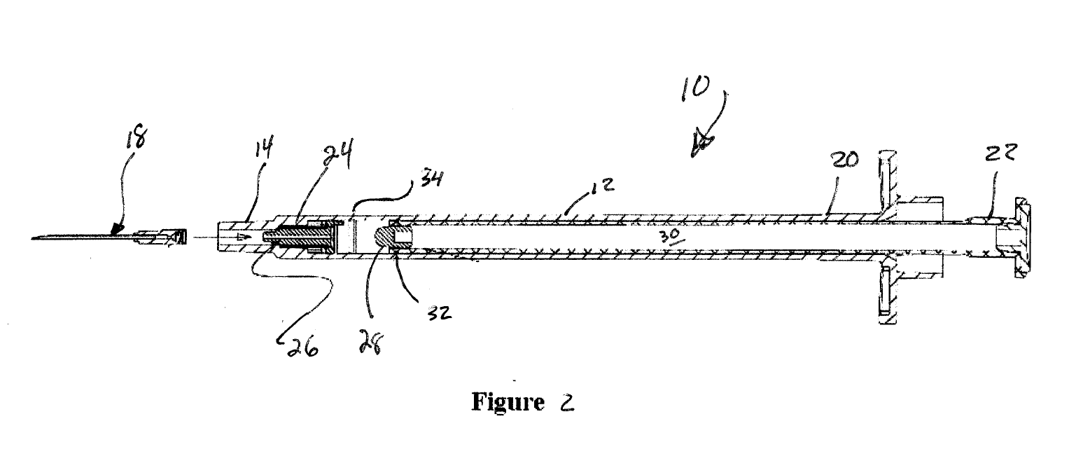

Referring to FIG. 1 there is shown a retractable needle syringe 10 having a syringe barrel 12 with a distal end 14 configured as a conical frustum tip. The conical frustum tip 14 is configured to attach to female Luer compatible devices including a filling needle & Luer hub 16 as shown, collectively referred to as a luer filling needle, as well as delivery tubes and the like. The syringe 10 is also shown to include a selectively attachable, interchangeable needle assembly 18 including a needle and threaded needle hub, allowing needles of different sizes and lengths to be interchanged with the syringe 10. Needle assembly 18 has radially extending ribs and is configured to be threadably coupled to a threaded needle stem 24 within the distal end of the conical frustum tip 14, as shown in the FIG. 2 as will be described shortly. Syringe 10 also includes a syringe proximal end 20 and a plunger 22 slidable therein from the proximal end, the plunger 22 configured to both aspirate a fluids through the Luer filling needle/hub 16, and also dispense the medicant upon compression. The plunger 22 is also configured to aspirate a fluid through the needle assembly 18 if desired. The filling needle/hub 16 may be desired as it is a common inexpensive needle that can also speed up the drawing process, and also prevents the possible unintentional retraction of the needle assembly 18 during insertion into the medicant vial or during the handling of the syringe 10 when drawing the medicant. Moreover, the conical frustum tip 14 advantageously allows the syringe 10 to be conveniently prefilled with medicant at one place and capped, then transported to a patient with or without needle assembly 18 as desired. This design is a significant advantage for many healthcare providers involved in the processing and handling of syringes until ultimate delivery of the medicant to a patient.

Referring to FIG. 2, there is shown a longitudinal cross sectional view of syringe 10 of FIG. 1, detailing the needle stem 24 having a threaded distal end 26 configured to receive the needle assembly 18. Notably, the needle stem 24 is positioned within the distal end of the conical frustum tip 14 and is advantageously protected from axial forces which could inadvertently being contacted and creating an unintended retraction of spring biased needle stem 24, such as when the needle assembly is secured to needle stem. Also shown is the plunger 22 having a plug 28 at a distal end thereof, which plug is in sealing arrangement with a cavity 30 of plunger 22 prior to retraction of needle assembly 18 therein, and which plug is dislodged into the cavity by the needle stem 24 that retracts with needle assembly 18 after an injection. The distal end of plunger 22 proximate the plug 28 has an integral seal 32 extending annularly thereabout. The interior surface of the syringe includes a plurality of annular detents 34 configured for positioning the seal 32 in a rested position, before the distal end of plunger 22 axially engages a protrusion 38 of an annular ring 36 coupled to the needle stem 24 by a breakable membrane, as more fully described in Applicant's U.S. Pat. No. 7,803,132 B1, the teaching of which are included herein by reference.

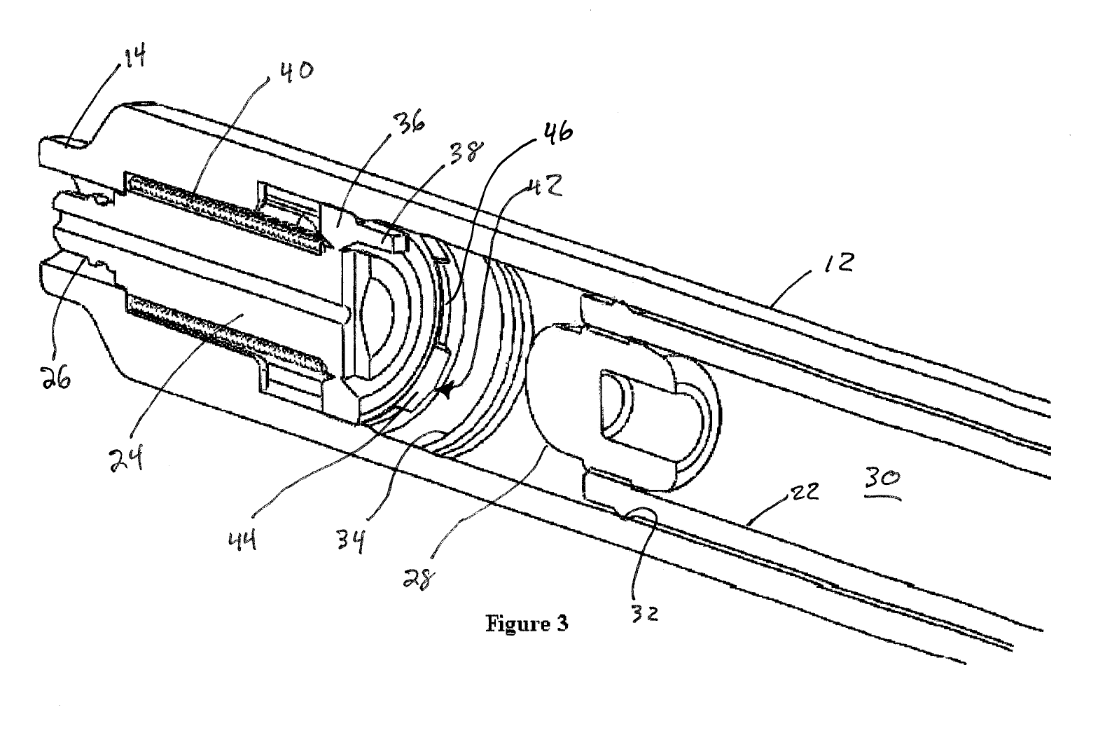

FIG. 3 depicts an enlarged cross sectional view of the needle stem 24, including the threaded needle distal end 26, positioned within the conical frustum tip 14, and coupled to the retaining ring 36 with protrusion 38 by the breakable membrane. Also shown is a spring 40 configured to retract the needle assembly 18 in response to the plunger 22 engaging the protrusion 38 after delivering the medicant to create a progressive separation of the retaining ring 36 from the needle stem 24 and the retraction of needle stem 24 into the plunger cavity 30. The annular detents 34 configured to seat position seal 32 are also shown.

This FIG. 3 also shows the interior surface of the barrel 12 including an annular retaining ledge generally shown at 42 configured to retain the needle stem 24 distally thereof such that the needle stem 24 is securely seated in the syringe barrel distal end, including during attachment of needle assembly 18, and until a complete retraction of the needle stem 24 and needle assembly 18 into cavity 30. Notably, the retaining ledge 42 is segmented, and is defined by segments of raised protrusions 44 extending from the interior barrel wall with notches or detents 46 defined between the protrusions 44 to provide several features. The segmented retaining ledge 42 enables reduced activation force required by the plunger to separate and break the retaining ring 36 from the needle stem 24, which provides an improved tactile feeling of the seal 32 and comfort of use. The segmented ledge 42 also makes it easier to install the needle stem 24 and retaining ring 36 in the barrel distal end, such that it snaps into place therepast with less axial force.

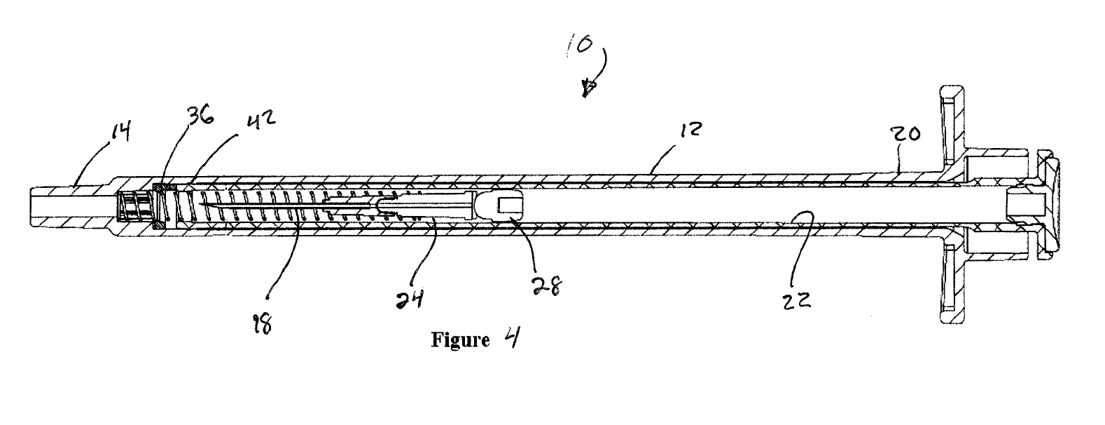

FIG. 4 depicts the needle stem 24 and needle assembly 18 after retraction into the plunger cavity 30.

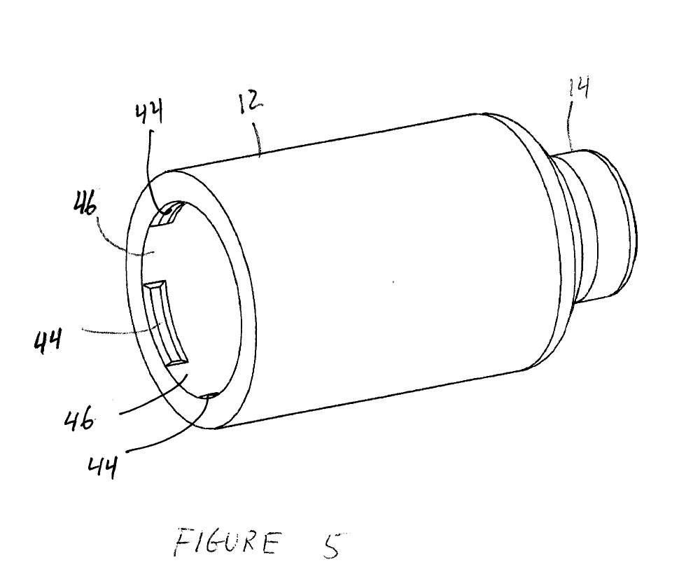

FIG. 5 depicts a cross section of the syringe barrel distal end taken proximate the segmented retaining ledge 42.

While the invention has been described in detail and with reference to a specific embodiment thereof, it will be apparent to one skilled in the art that various changes and modifications can be made therein without departing from the spirit and scope thereof. Thus, it is intended that the present invention covers the modifications and variations of this invention provided they come within the scope of the appended claims and their equivalents.

I claim:

1. A syringe, comprising:
a barrel having a proximal end and a distal end, and having a cavity configured chamber to hold a fluid;
a plunger disposed in the barrel and configured to advance toward the barrel distal and proximal ends;
a needle coupled to the barrel distal end and configured to deliver the medicant upon advancement of the plunger toward the barrel distal end, the needle further configured to be selectively attached and detached from the barrel distal end; and
the barrel distal end further comprising a conical frustum tip configured to couple to a Luer fitting to permit the syringe to draw the medicant there through, the conical frustum tip further configured to permit the needle to be interchangeable with the barrel distal end.

2. The syringe as specified in claim 1 wherein the conical frustum tip is further configured to communicate and form a liquid sealable arrangement with female Luer fittings.

3. The syringe as specified in claim 1 wherein the barrel distal end comprises a stem disposed in the barrel and configured to couple to the needle, the stem configured to selectively retract within the barrel upon delivery of the medicant from the barrel.

4. The syringe as specified in claim 3 wherein the stem is spring biased toward the barrel proximal end.

5. The syringe as specified in claim 3 wherein the stem is positioned closely proximate the distal end of barrel.

6. The syringe as specified in claim 4 wherein the stem is positioned within the conical frustum distal end.

7. The syringe as specified in claim 2 wherein the conical frustum tip is configured to permit the needle to axially extend there through.

8. The syringe as specified in claim 3 wherein the needle is configured to be selectively coupled to the stem.

9. The syringe as specified in claim 1 wherein the barrel distal end includes a breakable member configured to break and permit retraction of the needle into the barrel upon delivery of the medicant.

10. The syringe as specified in claim 3 wherein the barrel distal end includes a breakable member coupled to the stem and configured to break and permit retraction of the needle into the barrel upon delivery of the medicant.

11. The syringe as specified in claim 1 wherein the conical frustum tip is integral to the barrel.

12. The syringe as specified in claim 1 wherein the barrel distal end includes a collar at least partially covers the conical frustum tip.

13. The syringe as specified in claim 12 wherein the collar includes a threadable female connector.
